(19) United States
(12) Patent Application Publication
(10) Pub. No.: US 2010/0036240 A1
(43) Pub. Date: Feb. 11, 2010

(54) Multi-modality system for imaging in dense compressive media and method of use thereof

(76) Inventors: Aly M. Ismail, Irvine, CA (US); Kenneth Brian Cooper, La Canada, CA (US); Khaled N. Satama, Troy, NY (US)

Correspondence Address: LAW OFFICES OF ROBERT HART, 28 EAST JACKSON BUILDING, 10TH FLOOR, SUITE H528, CHICAGO, IL, 60604, US

(21) Appl. No.: 12/221,868

(22) Filed: Aug. 7, 2008

(51) Int. Cl. A61B 6/00; A61B 8/00

(52) U.S. Cl. 600/425; 600/437

(57) ABSTRACT

A multi-modality system and method for performing screening/detection, imaging and diagnosis/characterization of materials and objects in dense compressive media, such as in medical soft tissue applications, is disclosed. Medical tissue applications include but are not limited to the detection and diagnosis of breast tumors. Generally, the present invention involves coupling an ultrasound subsystem for exciting target tissues with a microwave subsystem for measuring the response, imaging and diagnosing the target tissues.

### CROSS REFERENCE TO RELATED APPLICATIONS

This application claims the benefit of provisional patent Ser. No. 60/070,003 filed Mar. 19, 2008.

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of Invention

The present invention relates generally to the field of imaging in dense compressive media, and more particularly to a novel system and method of use thereof for imaging in medical soft tissue applications such as dermatology, orthopedics and bone fractures, and breast tumor scanning/detection and diagnosis/characterization.

According to the U.S. National Library of Medicine and the National Institutes of Health, one in eight women will be diagnosed with breast cancer. One in sixteen women will die prematurely due to breast cancer. Breast cancer is more easily treated and often curable if it is discovered early. Breast cancer stages range from 0 to IV. The higher the stage number, the more advanced the cancer. According to the American Cancer Society (ACS), the 5-year survival rates for persons with breast cancer that is appropriately treated are as follows: 100% for Stage 0, 100% for Stage I, 92% for Stage IIA, 81% for Stage IIB, 67% for Stage IIIA, 54% for Stage IIIB, and 20% for Stage IV. Clearly, early detection is the primary factor in the successful treatment of breast cancer. Early breast cancer usually does not cause symptoms, therefore accentuating the importance of early detection devices and methods.

2. Discussion of Related Art

The usefulness of methods and/or devices to perform breast cancer detection is well recognized. A variety of related art methods and/or devices are directed to the problem. However, each related art method and/or device possesses significant disadvantages.

The principal methods of detecting breast cancer are clinical physical examination, self-examination, and X-ray mammography. Efforts have been made to develop alternative solutions to the problem of breast cancer detection and diagnosis, including magnetic resonance imaging (MRI) and microwave radar imaging.

In a clinical physical examination, a doctor performs a tactile physical examination of the breasts, armpits, and the neck and chest area. The physical examination is intended to discover lumps indicative of cancer. However, the clinical physical examination cannot identify the nature of the lump and lacks the sensitivity or resolution of other methods.

The breast self-examination is essentially the same as the clinical physical examination, but it is performed by the subject outside of the clinical environment. The breast self-examination is similar in benefit and limitation to the clinical physical examination.

X-ray mammography is currently the only FDA-certified early breast cancer screening technology. X-ray mammography, in some cases, can detect breast cancers before they can be detected by a physical examination. One breast at a time is rested on a flat surface that contains an X-ray detection media; typically a film exposure plate or a digital imaging modality such as semiconductor detectors. A device called a compressor is pressed firmly against the breast to flatten the breast tissue. This results in substantial discomfort to the patient. The patient holds her breath as a series of X-ray images are taken from several angles. Deodorant, perfume, powders and jewelry must be removed to prevent blockage of the X-rays. In each examination, the patient is exposed to destructive ionizing radiation, thus incurring a risk of realizing an induced breast tumor. X-ray mammography is considered a health risk for women who are pregnant or breast-feeding, and it is not recommended for women under the age of fifty. Further, X-ray mammography is a poor method for early-stage cancer detection. In a recent study, only 52 percent of high-grade ductal carcinoma in situ (DCIS), the form most likely to develop into invasive cancer, were detected by X-ray mammography. “MRI for Diagnosis of Pure Ductal Carcinoma In Situ: A Prospective Observational Study,” Christiane Kuhl, et. al., The Lancet, vol. 370, issue 9586, 11 Aug. 2007, pages 485-492.

Due to the limitations and disadvantages in these current methods, there exists an on-going search for other effective methodologies. Magnetic resonance imaging (MRI) and microwave radar are two solutions of interest.

Magnetic resonance imaging (MRI) employs powerful magnets and radio waves to generate images inside the body. The magnetic field produced by an MRI is about ten thousand times greater than the Earth's magnetic field. The magnetic field polarizes the magnetic moment of hydrogen atoms in the body. When properly tuned radio waves are then transmitted through the body, they are differentially absorbed depending on the types of tissue encountered. The resulting radio signal can thus often distinguish healthy versus cancerous tissue. MRI represents a substantial improvement over X-ray mammography in terms of early screening, detecting 98 percent of high-grade DCIS compared with 52 percent detection by X-ray mammography, as noted in Kuhl, et al., “MRI for Diagnosis of Pure Ductal Carcinoma In Situ: A Prospective Observational Study,” Christiane Kuhl, et. al., The Lancet, vol. 370, issue 9586, 11 Aug. 2007, pages 485-492.

While MRI offers improved screening accuracy over X-ray mammography and eliminates the risk associated with ionizing radiation, it also has significant disadvantages. Many patients find the MRI procedure uncomfortable. The patient may be required to fast from four to six hours prior to the scan. Then, the patient lies on a narrow table which slides into the middle of the MRI scanner. The MRI machine may induce anxiety in patients with a fear of confined spaces. Further, the MRI machine produces loud percussive and buzzing noises which may be disconcerting to the patient. Finally, because several sets of images are required, each taking from two to fifteen minutes, the patient must be exposed to the MRI environment for an hour or longer. The patient is required to lie motionless for this long period of time because excessive movement can blur MRI images and cause errors. In addition, because the magnet is very strong, certain types of metal can cause significant errors in the images, and the strong magnetic fields created during an MRI can interfere with certain medical implants. Persons with pacemakers or other metallic objects in the body, such as ear implants, brain aneurism clips, artificial heart valves, vascular stents and artificial joints should not be exposed to MRI. Finally, the high cost of procuring and operating an MRI machine, and the lack of technicians skilled in reading breast MRIs present additional disadvantages to its use.

Research has turned to consideration of non-invasive ultrasound methods for screening and diagnosis, utilizing acoustic means for both excitation of the tissues and for measurement and imaging of the excited tissues. A publication by Alizad, et al., discusses one such acoustic method wherein a hydrophone is employed to detect the acoustic waves generated by the motion induced in the tissue. The detected acoustic waves are processed into imaging information. A. Alizad, M. Fatemi, L. E. Wold and J. F. Greenleaf, “Performance of Vibro-Acoustography in Detecting Microcalcifications in Excised Human Breast Tissue: A Study of 74 Tissue Samples,” IEEE Trans. Med. Imaging., vol. 23, pp. 307-312, March 2004. Hynyen, et al., U.S. Pat. No. 6,984,209 discloses another acoustic method which incorporates a pulse-echo ultrasound transceiver to perform the measurement and imaging function. Methods that rely upon acoustic measurement alone are disadvantaged by noise, contrast and speckle limitations, and by the necessity to trade off low-frequency penetration against high-frequency resolution.

Microwave detection methods offer a factor of five improvement in detection sensitivity and diagnostic capacity over ultrasound methods. Microwave transmission is very sensitive to variations in media material permittivity, which may vary by a factor of five, while ultrasound sensitivity to these permittivity variations is less than ten percent. J. E. Joy, E. E. Penhoet and D. B. Petitti, “Saving Women's Lives: Strategies for Improving Breast Cancer Detection and Diagnosis,” Institute of Medicine and National Research Council, ISBN: 0-309-53209-4, 2005. Ultrasound methods rely on the measurement of variations in the mechanical properties of benign tissue and cancerous tumors, which are not large. On the other hand, microwave methods take advantage of the difference in dielectric constants associated with the water content of benign tissue and cancerous tumors, which vary dramatically. Therefore, research is turning to consideration of microwave radar devices and methods for soft tissue imaging. Microwave imaging offers a low-stress, low risk solution; requiring short exposure periods without the dangers or discomforts associated with X-ray mammography or MRI. The scientific principles are defined and experimental demonstration discussed in a publication by Li, et al., “Microwave Imaging via Space-Time Beamforming: Experimental Investigation of Tumor Detection in Multilayer Breast Phantoms,” Xu Li, et al., IEEE Transactions on Microwave Theory and Techniques, Vol. 52, No. 8, August 2004. Li, et al., experimentally demonstrated the effectiveness of radar imaging principles in breast tumor detection applications employing a two-dimensional scanning methodology to synthesize a two dimensional antenna array. However, imaging methods that rely on microwave alone are disadvantaged by the necessity to trade off low-frequency penetration against high-frequency resolution.

Because of the limitations associated with each individual screening, imaging and diagnosis method, research is considering combining multiple imaging modalities. Rosner, et al., U.S. Patent Application No. 2007/0276240 discloses a system which uses both acoustic and microwave methods for imaging. The ultrasound subsytem transmits ultrasound waves into the target and receives the echo. The microwave subsystem transmits a microwave signal into the target and receives the reflection. The ultrasound and microwave modalities operate independently. This simple integration of two modalities does not take advantage of the physical interaction of the ultrasound and microwave modalities. Therefore, this combined system possesses the disadvantages of each subsystem. It is difficult to concurrently achieve high penetration, high resolution, fast scanning and high contrast using either subsystem alone.

### BRIEF SUMMARY AND OBJECTS OF THE INVENTION

In view of the foregoing disadvantages inherent in the known devices and methods in the related art, the present invention provides a novel multi-modality system and method for performing screening/detection, imaging and diagnosis/characterization of materials and objects in dense compressive media, particularly but not exclusively in medical soft tissue applications. Specifically, the present invention involves coupling an ultrasound subsystem for stimulating target tissues with a microwave subsystem for measuring the response of the stimulated target tissues. The present invention involves a true hybrid integration of the ultrasound and microwave modalities, taking advantage of the best attributes of each subsystem modality. The superior resolution and focus characteristics of high-frequency ultrasound input waves are employed to excite Doppler displacements of materials in the target breast. At the same time, the superior penetration and high diagnostic contrast capabilities of the microwave modality are employed to perform the diagnosis an imaging function. The present invention enhances early detection and diagnosis capability without the disadvantages of the related art systems and methods.

Low cost is achieved by enabling application of low-cost components, such as compact radio frequency components developed for the wireless communications industry and existing ultrasound application components.

In one embodiment of the present invention, the complexity, cost and time associated with mechanical scanning is avoided by employing an ultrasound transducer array in place of scanning ultrasound transducers.

The present invention enables achievement of a small form factor, relative to MRI and X-ray devices, to reduce cost and enhance flexibility and convenience.

The present invention enables three-dimensional detection and diagnosis imaging. In one alternative embodiment of the present invention, ultrasound and microwave subsystem combinations are implemented in multiple axes. These multi-axis subsystems cooperate to provide superior three-dimensional imaging capability. In yet another embodiment, phased array operation of the ultrasound subsystem allows mapping of two-dimensional planes of varying depths within the target breast. These two-dimensional maps may be integrated to create three-dimensional images.

The present invention minimizes patient discomfort attendant to related art systems and methods. The present invention requires merely soft compression to maintain contact between the target breast and the ultrasound transducer and microwave antenna, eliminating the discomfort associated with X-ray mammography breast compression. In addition, stress associated with ionizing radiation exposure is eliminated. Further, the relatively simple apparatus and short imaging time enabled by the present invention eliminates the discomfort associated with long exposure to the confining and noisy environment of MRI apparatuses.

The present invention eliminates health risks associated with related art systems and methods. The present invention eliminates the risk of short-term or long-term deleterious affects associated with ionizing radiation exposure in X-ray mammography, and the risks associated with exposure to powerful magnetic fields in MRI.

Other advantages of the present invention will become readily apparent to those with skill in the art from the following figures, descriptions and claims. As will be appreciated by those of skill in the art, the present invention may be embodied as an apparatus, systems or methods. It is intended that such other advantages embodied as other apparatus, systems and methods be included within the scope of this invention, and the examples set forth herein shall not be limiting.

### BRIEF DESCRIPTION OF THE DRAWINGS

The nature of this invention, as well as all its objects and advantages, will become readily apparent and understood upon reference to the following detailed description when considered in conjunction with the accompanying drawings, in which like reference numerals designate like parts throughout the figures thereof, and wherein:

FIG. 1 shows the orientation of the system with respect to the patient and the imaging target breast in one preferred embodiment of the present invention.

FIG. 2 provides a schematic representation of the ultrasound subsystem.

FIG. 3 provides a schematic representation of the microwave imaging subsystem.

FIG. 4 shows the microwave transmission into the target breast, and the resultant display of the reflected microwaves, prior to activation of the ultrasound subsystem.

FIG. 5 shows the ultrasound wave transmission through the subject breast, the resultant displacement of the target tumor, and the display of the reflected microwaves resulting from the ultrasound stimulation of the tumor.

FIG. 6 presents an alternative embodiment of the ultrasound subsystem featuring an ultrasound transducer array.

FIG. 7 presents an alternative embodiment of the ultrasound subsystem featuring a focused ultrasound wave pulse.

FIG. 8 presents an alternative embodiment of the present invention employing paired ultrasound transducers and microwave antennas in multi-axis orientations.

FIG. 9 shows an alternative embodiment of the present invention implementing both the ultrasound transducer and the microwave antenna on the same side of the subject breast.

FIG. 10 presents alternative scanning approaches.

### DETAILED DESCRIPTION OF THE PREFERRED EMBODIMENTS

The following description is provided to enable any person skilled in the art to make and use the invention and sets forth the best modes contemplated by the inventor of carrying out his invention. Further, while a breast is used in the description of these embodiments, it is to be noted that any turbid medium may be processed with this invention. Thus the present invention shall not be limited to the examples disclosed. The scope of the invention shall be as broad as the claims will allow.

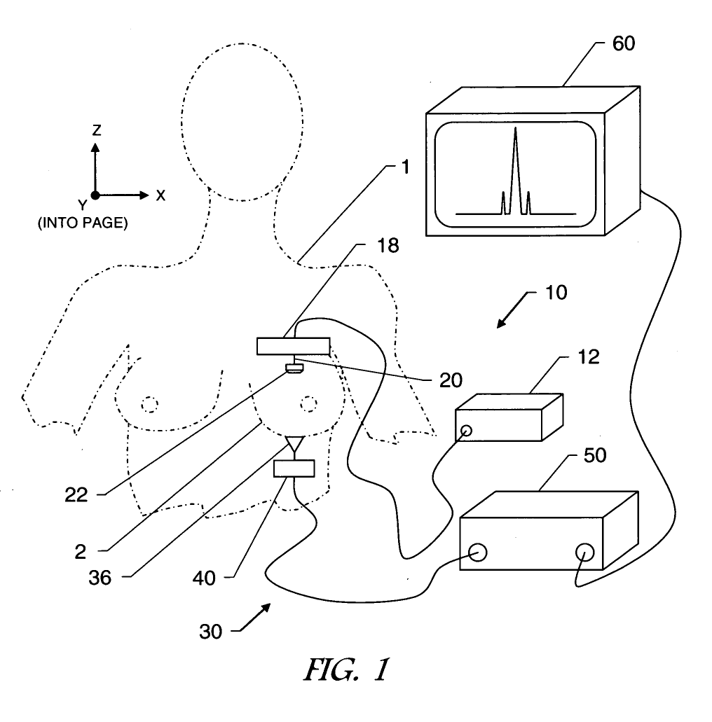

Referring now to the drawings, FIG. 1 shows the orientation of the system with respect to the patient 1 and the imaging target breast 2 in one preferred embodiment of the present invention. An ultrasound subsystem 10 and a microwave imaging subsystem 30 are employed in combination to detect and diagnose tumors in the breast 2. An ultrasound transducer 22 and a microwave antenna 36 are oriented with respect to the target breast 2 of the patient 1. In one preferred embodiment, the ultrasound transducer 22 is oriented along the same axis, the Z-axis, as the microwave antenna 36: the ultrasound transducer 22 aimed in the negative z direction and the microwave antenna 36 aimed in the positive Z direction. A radio frequency transceiver 40 generates and transmits microwave signals to the microwave antenna 36. The microwave antenna 36 transmits microwaves into the target breast 2. Reflected microwaves are collected by the microwave antenna 36 and received by the radio frequency transceiver 40. A computer/signal and data processor 50 containing signal processing circuitry and data processing algorithms processes the output of the radio frequency transceiver 40 and sends the resultant data to the display 60 for access by the technician. The display 60 may be a video screen, a printing device, a photographic device, an oscilloscope, a spectrum analyzer, or any useful medium for communicating system output to the technician. The data may be usefully represented as individual spectra, one-dimensional line scans, two-dimensional cross-sectional constructions, or volume images. A scan controller/actuator 18 working in combination with a mechanical actuator 20 orients the ultrasound transducer 22 to enable scanning of the entire target breast 2. An ultrasound electronics assembly 12 generates and transmits ultrasound waves to the ultrasound transducer 22. The ultrasound transducer transmits the ultrasound waves to the target breast 2 to stimulate the tissues therein.

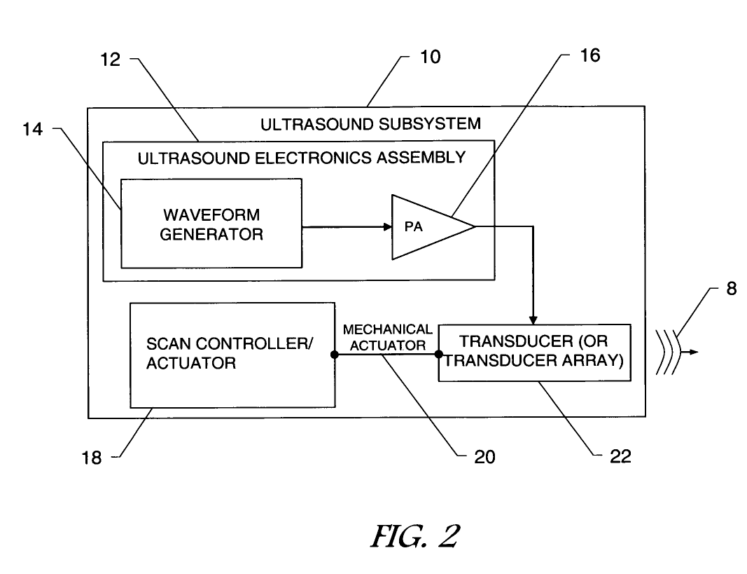

FIG. 2 provides a schematic representation of the ultrasound subsystem 10. An ultrasound electronics assembly 12 is shown housing a waveform generator 14 and a power amplifier 16. The waveform generator 14 produces an input ultrasound waveform. The power amplifier 16 conditions the input ultrasound waveform and transmits said ultrasound waveform to the ultrasound transducer 22. The ultrasound transducer 22 transmits the amplified input ultrasound wave 8 into the target breast 2. To maximize transmission of the ultrasound wave 8 into the breast, an ultrasound conductive gel may be used at the interface of the ultrasound transducer 22 and the target breast 2. In a preferred embodiment of the present invention, the ultrasound transducer 22 must be physically relocated to perform a scan of the entire breast 2. This scanning function is performed by a scan controller/actuator 18 working in combination with a mechanical actuator 20.

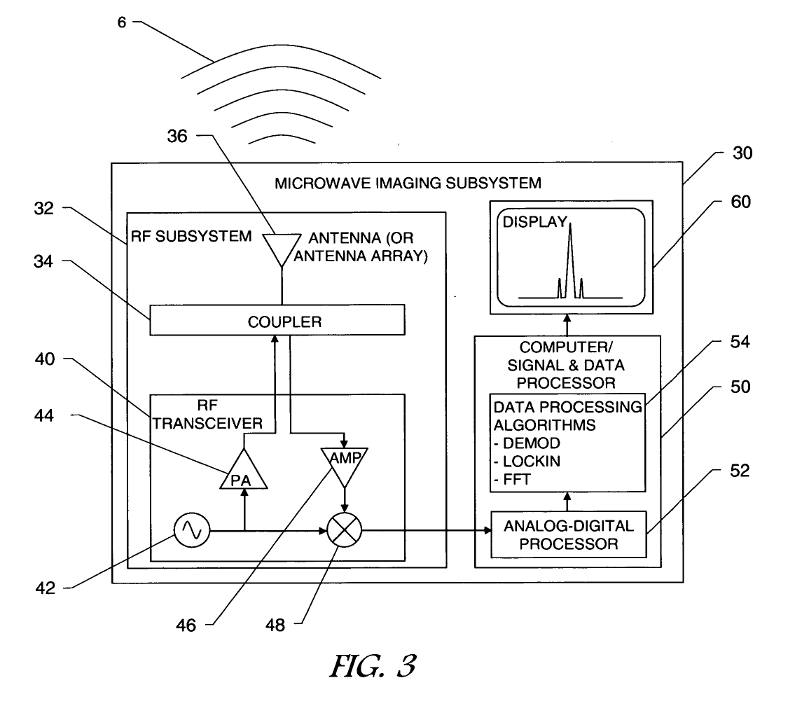

FIG. 3 provides a schematic representation of the microwave imaging subsystem 30 comprising an RF subsystem 32, a computer/data processor 50 and a display 60. The RF subsystem 32 comprises an RF antenna 36, a coupler 34, and an RF transceiver 40. The RF transceiver 40 comprises a waveform generator 42, a power amplifier 44, an amplifier 46 and a mixer 48. The waveform generator 42 produces an input waveform. The power amplifier 44 conditions the input waveform and transmits said waveform through the RF coupler 34 to the RF antenna 36. The RF antenna 36 transmits the microwave 6 into the target breast 2. To efficiently transmit the microwave 6 to the breast 2, the RF antenna 36 is in physical contact with the breast 2. In a preferred embodiment of the present invention, the RF antenna 36 is made from a material that closely matches the dielectric constant of the breast 2. In an alternative embodiment, a dielectrically loaded antenna, in which the antenna 36 is embedded in a material that matches the dielectric constant of the breast 2, may be employed to reduce reflections. Due to the wide propagation angle of the microwave 6 in the breast 2, it is not necessary to move the RF antenna to scan the breast 2. However, an alternative embodiment of the present invention may employ an RF antenna 36 scanning means, if desired. Microwaves reflected by normal/cancerous tissue boundaries and/or inclusions are collected by the microwave antenna 36 and transmitted through the coupler 34 to an amplifier 46. Input waveforms from the waveform generator 42 and reflected microwaves from the amplifier 46 are passed through a mixer 48 and conveyed to an analog-digital processor 52. Data processing algorithms 54 such as demodulation, and lockin detection or fast Fourier transform algorithms operate on the digital data from the analog-digital processor 52. The resultant frequency and power data is transmitted to a display 60 for viewing by the technician.

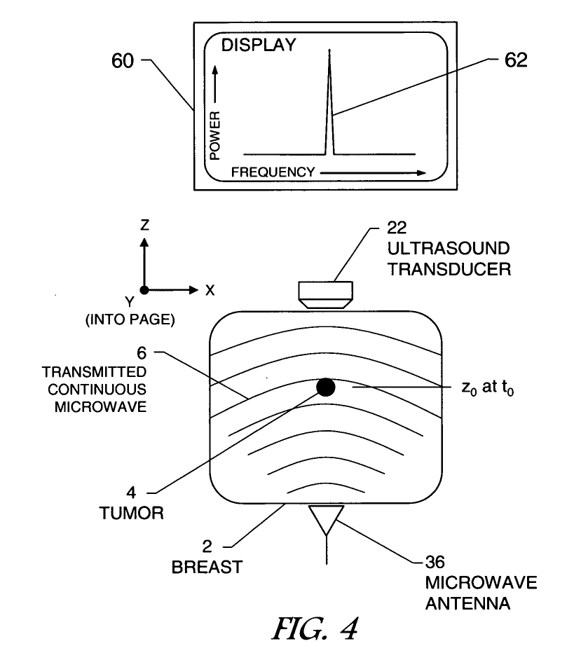

FIG. 4 shows the microwave 6 transmission into the target breast 2, and the resultant display of the reflected microwaves, at time t₀ prior to activation of the ultrasound subsystem 10. In one preferred embodiment of the present invention, a continuous microwave 6 is employed. It is anticipated that other input waveforms and methods, such as frequency modulation and pulse-delay, may be usefully employed to reduce clutter signals and improve the probability of tumor detection. The microwave 6 is transmitted by the RF antenna 36 into the breast 2. Prior to activation of the ultrasound subsystem 10, microwaves will be reflected back to the RF antenna 36 from the internal boundaries of the breast and from inclusions in the breast 2 such as a tumor 4. The reflected microwaves will be of the same frequency as the transmitted input microwaves 6. The reflected microwave will appear on the display 60 as a power spike 62 at the frequency of the transmitted wave. No position or shape information of the tumor 4 is detectable prior to activation of the ultrasound subsystem 10.

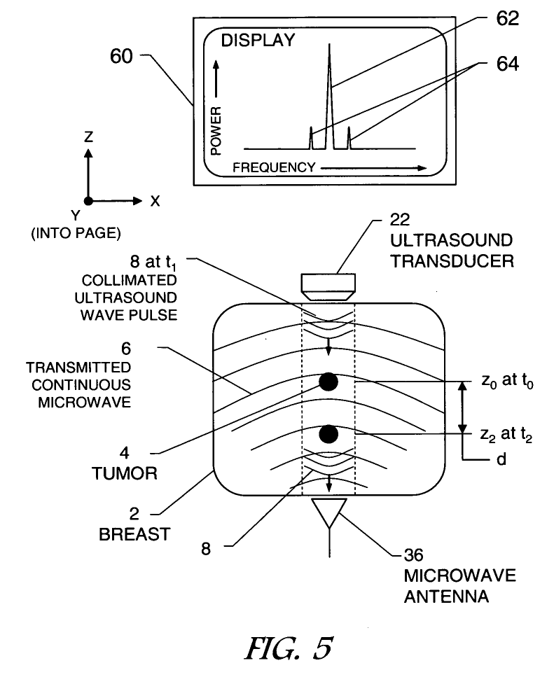

FIG. 5 shows the ultrasound wave 8 transmission through the subject breast 2, the resultant displacement of the target tumor 4, and the spectral representation of the reflected microwaves 6 resulting from the ultrasound stimulation of the tumor 4. At time t₀, no ultrasound waves have been transmitted into the breast 2. The tumor 4 is at rest at location z₀. A continuous microwave is transmitted into the breast 2 and reflections from the boundary of the breast 2 and the tumor 4 are displayed as a power spike 62 at the same fundamental frequency as that of the input microwave 6. At time t₁, an ultrasound wave 8 is introduced into the breast 2. In one preferred embodiment of the present invention, the ultrasound transducer 22 lens is designed to create a collimated ultrasound wave 8 which propagates essentially in a column through the breast 2. The ultrasound wave 8 travels at a significantly lower rate of speed than the microwave 6. At time t₂, the ultrasound wave impacts the tumor 4 and displaces said tumor 4 to location z₂. As the ultrasound wave passes the tumor 4, the tumor oscillates between location z₂ and z₀ before coming to rest again at essentially the initial location z₀. As the tumor 4 oscillates between position z₀ and position z₂, the Doppler effect results in a shift in the frequency of the reflected microwave. These frequency shifts appear on the display 60 as frequency sidebands 64. Presence of these sidebands indicates the presence of a tumor 4. The sidebands 64 are short lived, essentially lasting for the duration of the ultrasonic pulse passing through the tumor 4.

The power of the sidebands is determined through displacement analysis. If a signal is reflected off of a target whose range is changing with time according to r(t)=r₀+Δr(t), the received signal can be written as:

s(t)=cos [ω_{c}t+2π−Δr(t)/λ+φ₀]

Where ω_{c} is the carrier frequency and φ₀ is the phase

For a small-amplitude oscillation of a target with a displacement d and a modulation frequency fm, the range is given by:

Δr(t)=d sin(ωₘt)

And thus the signal becomes

s(t)=cos [ω_{c}t+2π−(d/λ)sin(ωₘt)+φ₀]

For d<<λ, this expression is simply the narrowband FM situation:

$$\begin{matrix}f ( t ) = cos [ {ω}_{c} t + ( d \text{/} λ ) sin ( {ω}_{m} t ) ] \\ = cos ( {ω}_{c} t ) cos ( ( d \text{/} λ ) sin ( {ω}_{m} t ) ) - sin ( {ω}_{c} t ) sin ( ( d \text{/} λ ) sin ( {ω}_{m} t ) ) \\ = cos ( {ω}_{c} t ) - ( d \text{/} 2 λ ) [ cos ( {ω}_{c} t - {ω}_{m} t ) - cos ( {ω}_{c} t + {ω}_{m} t ) ]\end{matrix}$$

Each sideband is smaller than the carrier by:

P_{sideband}=10 log(d²/4 λ²)=20 log(πf_{c}d/c) dBc.

Radio frequency sensitivity is determined by the equation:

Sensitivity=NF+KT+10 log(BW)+SNR-10 log(Average)

### Where

NF: The receiver input referred noise figure (Typically 3-5 dB)

KT: Thermal noise power density (−174 dBm/Hz)

BW: Receiver noise bandwidth in Hz (typically1-2 MHz)

SNR: Required detector SNR in dB (20 dB)

Average: Coherently collected samples over sample time

If sensitivity is not sufficient, and to give system sensitivity a boost, a continuous wave may be employed such that:

Sensitivity=NF+KT+10 log(BW)+SNR-10 log(Average)−10 log(gain)

### Where

gain: gain achieved due to applying continuous wave

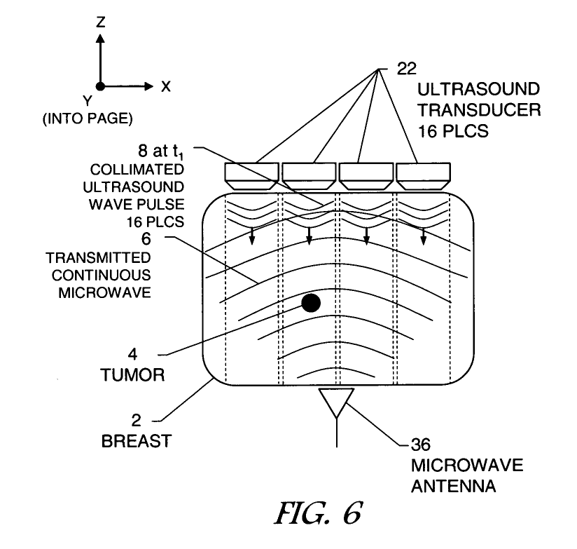

FIG. 6 prevents an alternative embodiment of the ultrasound subsystem 10 featuring an array of ultrasound transducers 22. Use of an array of transducers 22 is an alternative to scanning with a single ultrasound transducer 22. In this alternative, the design and operational complexities of a scanning system are traded against the design and operational complexity of a fixed array. A 4×4 array is shown for illustrative purposes. It is obvious and anticipated that arrays of various sizes may be usefully employed.

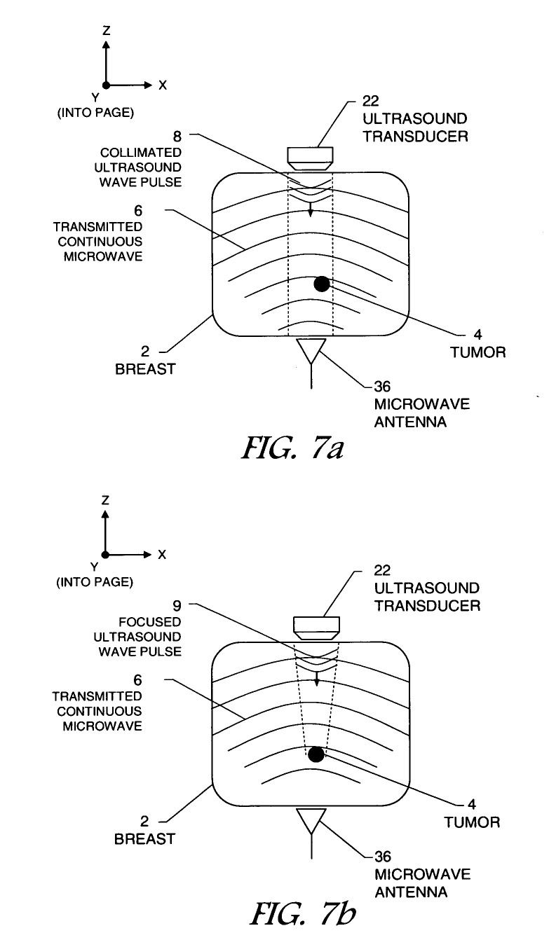

FIG. 7 presents an alternative embodiment and operation of the ultrasound subsystem 10. FIG. 7a shows the ultrasound transducer 22 designed to generate a collimated ultrasound wave 8. This configuration is used to perform the detection function. Upon detection of a tumor 4, an ultrasound transducer 22 designed to generate a focused ultrasound wave 9 is located such that the focal point of the ultrasound wave 9 is concentrated on the tumor 4 as shown in FIG. 7b. This configuration is used to perform the diagnosis function, enabling higher resolution definition of tumor size and shape, and the presence of multiple tumors.

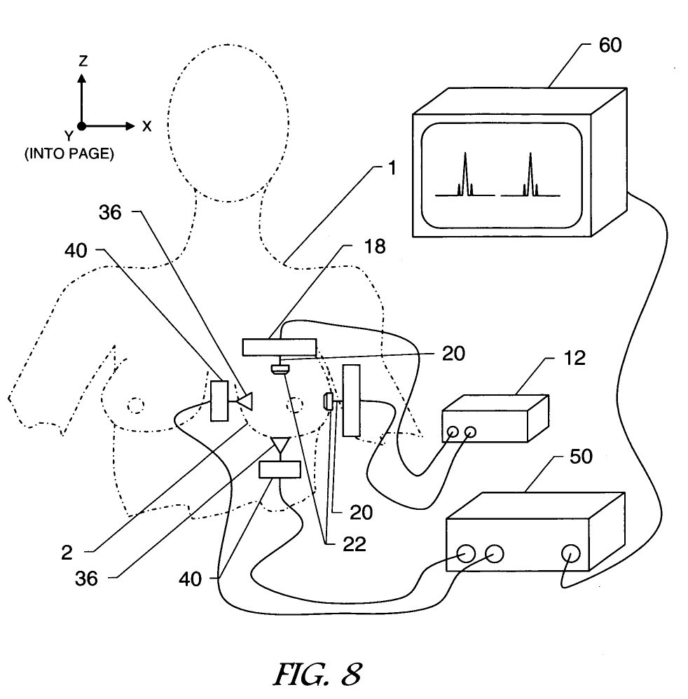

FIG. 8 presents an alternative embodiment of the present invention employing paired ultrasound transducers 22 and microwave antennas 36 in multi-axis orientations. This configuration enhances detection of multiple tumors, particularly in the case where one or more tumors would be in the shadow of another tumor in a single-axis detection configuration. For illustrative purposes, one transducer 22/antenna 36 pair is shown oriented along the Z-axis working in combination with another transducer 22/antenna 36 pair oriented along the X-axis.

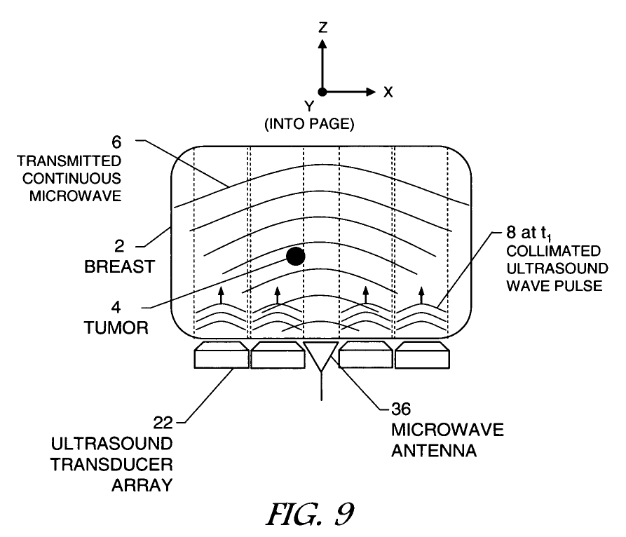

FIG. 9 shows an alternative embodiment of the multi-modality imaging system implementing both the ultrasound transducer 22 and the microwave antenna 36 on the same side of the subject breast 2. For illustrative purposes, a single microwave antenna 36 is depicted with an array of ultrasound transducers 22. An alternative embodiment is to replace the array of ultrasound transducers 22 with a single ultrasound transducer 22 which is actuated to scan the target breast 2. These alternative configurations enable simplified apparatus design.

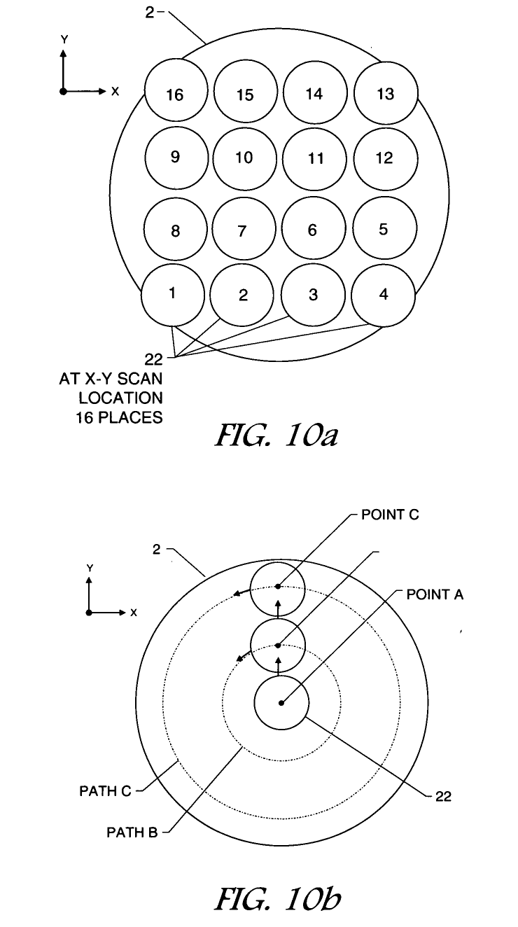

FIG. 10 presents alternative scanning approaches. FIG. 10a illustrates a representative x-y planar scanning scheme wherein the ultrasound transducer 22 is moved sequentially from station to station in the X-Y plane as shown for an exemplary 4×4 scanning matrix. In this scheme, the ultrasound transducer 22 transmits ultrasound waves 8 at each sequential location. FIG. 10b illustrates an alternative scanning scheme wherein the ultrasound transducer 22 is transmitting continuously as it is moved in circular paths of increasing diameter. In this example, the ultrasound transducer 22 begins operation at point A, is indexed to point B, follows path B, then is indexed to point C and follows path C. Other continuous-scan patterns may be employed, such as moving the ultrasound transducer 22 in a continuously-increasing spiral pattern in the X-Y plane.

Various embodiments of the present invention may be exercised in ways other than those illustrated in the examples shown in the Figures. Such alternative embodiments are within the contemplation of the present invention. The examples are not intended to limit the scope of this invention, which shall be as broad as the claims will allow.

In addition, the present invention may be adapted to a variety of applications in both medical and non-medical fields. The field of medical soft tissue imaging includes orthopedics, dermatology, breast tumor screening/detection, imaging and diagnosis/characterization, and other medical applications. Such alternative applications are within the contemplation of the present invention and the scope of the invention shall be as broad as the claims allow.

The physical implementation of the present invention may be varied without departing from the spirit of the invention. Elements and components may be implemented, added, interchanged, combined and/or packaged in a variety of embodiments. Various changes may be effected in structure, design, choice of components and materials, etcetera without departing from the spirit of the present invention. Such alternative embodiments, elements and implementations are within the contemplation of the present invention and the scope of the invention shall be as broad as the claims allow.

Accordingly, the scope of the invention should be determined not by the embodiments illustrated, but by their legal equivalents, and shall be as broad as the claims will allow.

The following references are of utility in understanding the foregoing specification, and are incorporated herein by reference:

Christiane Kuhl, et. al., “MRI for Diagnosis of Pure Ductal Carcinoma In Situ: A Prospective Observational Study,” The Lancet, vol. 370, issue 9586, 11 Aug. 2007, pages 485-492.

Li, Xu, et. al. (2004): “Microwave Imaging via Space-Time Beamforming: Experimental Investigation of Tumor Detection in Multilayer Breast Phantoms,” IEEE Transactions on Microwave Theory and Techniques, Vol. 52, No. 8, pp 1856-1865.

Nanda, R. (2007): “Breast Cancer,” Medline Plus Medical Encyclopedia, the U.S. National Library of Medicine and the National Institute of Health, <http://www.nlm.nih.gov/medlineplus/ency/article/000913.htm>.

A. Alizad, M. Fatemi, L. E. Wold and J. F. Greenleaf, “Performance of Vibro-Acoustography in Detecting Microcalcifications in Excised Human Breast Tissue: A Study of 74 Tissue Samples,” IEEE Trans. Med. Imaging., vol. 23, pp. 307-312, March 2004.

J. E. Joy, E. E. Penhoet and D. B. Petitti, “Saving Women's Lives: Strategies for Improving Breast Cancer Detection and Diagnosis,” Institute of Medicine and National Research Council, ISBN: 0-309-53209-4, 2005.

C. Maleke, J. Luo and E. E. Konofagou, “2D Simulation of the Harmonic Motion Imaging (HMI) With Experimental Validation,” IEEE Ultrasonics Symposium, pp. 797-800, 2007.

E. E. Konofagou, M. Ottensmeyer, S. L. Dawson and K. Hynynen, “Harmonic Motion Imaging—Applications in the Detection of Stiffer Masses,” IEEE Ultrasonics Symposium, pp. 558-561, 2003.

Reinberg, Steven (Aug. 10, 2007): “MRI Beats Mammograms at Spotting Early Breast Cancer,” HealthDay News <http://www.healthday.com/Article.asp?AID=607199>.

What is claimed is:

1. A system for screening, diagnosing and imaging of materials and objects in a dense compressive media comprising:
(a) an ultrasound means for exciting regions, materials and objects in the dense compressive media employing ultrasound waves; and
(b) a microwave means for detecting, characterizing and imaging said excited materials and objects.

2. A method for screening, diagnosis and imaging of materials and objects in a dense compressive media comprising the steps of:
(a) exciting regions, materials and objects in the dense compressive media employing ultrasound means; and
(b) screening, diagnosing and imaging the excited materials and objects employing microwave means.

3. The system according to claim 1, wherein
(a) said ultrasound means comprises:
(i) a means for generating input ultrasound waves; and
(ii) a means for transmitting said input ultrasound waves into the dense compressive media; and
(b) said microwave means comprises:
(i) a means for generating input microwaves;
(ii) a means for transmitting said input microwaves into the dense compressive media;
(iii) a means for detecting microwaves reflected by boundaries, materials and objects in the dense compressive media;
(iv) a means for processing detected microwaves into information describing the presence, location and characteristics of the materials and objects; and
(v) a means for communicating and displaying said information.

4. A system for screening, diagnosis and imaging of materials and objects in a dense compressive media comprising:
(a) an ultrasound subsystem for generating input ultrasound waves for exciting materials and objects in the dense compressive media; and
(b) a microwave imaging subsystem for screening, diagnosis and imaging said excited materials and objects.

5. A system for screening, diagnosis and imaging of materials and objects in a dense compressive media comprising:
(a) an ultrasound subsystem further comprising:
(i) a waveform generator to produce ultrasound waveforms;
(ii) a power amplifier to condition the generated ultrasound waveforms;
(iii) an ultrasound transducer to transmit the conditioned ultrasound waves into the target media and excite materials and objects within the media, and
(iv) a scan controller/actuator to enable scanning of the media; and
(b) a microwave imaging subsystem further comprising:
(i) a microwave generator for producing microwaves;
(ii) a power amplifier to condition the generated microwaves;
(iii) a microwave antenna to transmit the conditioned microwave into the target media;
(iv) a microwave antenna to detect microwaves reflected by media boundaries and materials and objects within the media;
(v) a computer/signal and data processor for processing detected analog microwave signals into information describing the presence, location and characteristics of the excited materials and objects; and
(vi) a display for communicating the information.

6. A method for screening, diagnosis and imaging of materials and objects in a dense compressive media comprising the steps of:
(a) generating input microwaves;
(b) transmitting said input microwaves into the dense compressive media;
(c) generating input ultrasound waves;
(d) transmitting said input ultrasound waves into the dense compressive media to excite materials and objects in the media;
(e) detecting microwaves reflected by the excited materials and objects;
(f) converting said detected microwaves into information describing the presence, location and characteristics of the excited materials and objects; and
(g) displaying said information.

7. The system according to claim 5, further comprising a plurality of ultrasound transducers or an ultrasound transducer array to excite the entire area of the dense compressive material without the need for mechanical scanning.

8. The system according to claim 5, further comprising a plurality of microwave antennas or a microwave antenna array to transmit microwaves into the dense compressive media.

9. The system according to claim 5, further comprising a plurality of microwave antennas or a microwave antenna array to detect microwaves reflected by media boundaries and materials and objects within the media;

10. The system according to claim 5, wherein a plurality of ultrasound transducer/microwave antenna combinations are employed in multiple axes to develop three-dimensional information.
